(19) United States
(12) Patent Application Publication
(10) Pub. No.: US 2023/0400459 A1
(43) Pub. Date: Dec. 14, 2023

(54) GARGLE SPECIMEN SAMPLING

(71) Applicant: HERO SCIENTIFIC LTD., Jerusalem (IL)

(72) Inventor: Robert Eric LEVITZ, Beit Shemesh (IL)

(73) Assignee: HERO SCIENTIFIC LTD., Jerusalem (IL)

(21) Appl. No.: 18/079,528

(22) Filed: Dec. 12, 2022

Related U.S. Application Data
(60) Provisional application No. 63/350,521, filed on Jun. 9, 2022.

(51) Int. Cl. G01N 33/569; G01N 33/543; G01N 33/487

(52) U.S. Cl. CPC G01N 33/56905; G01N 33/54388; G01N 33/56983; G01N 33/56944; G01N 33/487; G01N 2800/00; G01N 2333/005; G01N 2333/37; G01N 2333/195; G01N 2333/44

(57) ABSTRACT

A method is provided that includes rinsing an oral cavity of a subject with a rinse liquid. Thereafter, a gargle liquid is introduced into the oral cavity and gargled by the subject. Thereafter, at least a portion of the gargled liquid is collected as a specimen sample. Other embodiments are also described.

### CROSS-REFERENCE TO RELATED APPLICATIONS

The present application claims the benefit of U.S. Provisional Application 63/350,521, filed Jun. 9, 2022, which is incorporated herein by reference and assigned to the assignee of the present application.

### FIELD OF THE APPLICATION

Applications of the present invention relate to collecting samples.

### BACKGROUND OF THE APPLICATION

Streptococcal pharyngitis, streptococcal tonsillitis, or streptococcal sore throat (known colloquially as strep throat) is a type of pharyngitis caused by group A beta hemolytic streptococcus bacteria. Common symptoms include fever, sore throat, and enlarged cervical lymph nodes.

Known sampling methods for collecting a respiratory specimen sample include nasopharyngeal swabbing to sample the posterior nasal passages and the nasopharynx, nasal washes and aspirations to sample deeply into the nasal cavity, and throat swabbing to sample the pharynx. These procedures are often uncomfortable for the patient, require a healthcare professional to perform the sampling procedure, and expose the healthcare worker and others to contaminating particles.

Patients can self-collect alternative sample types such as gargled liquid, nasal washes, and saliva. However, these sample types tend to decrease test sensitivity compared to swab samples and are thus are not currently in widespread use.

PCT Publication WO 2018/158768 to Fruchter et al. describes a method for testing for presence of a particulate selected from the group consisting of: a microorganism, a fungus, a bacteria, a spore, a virus, a mite, a biological cell, a biological antigen, a protein, a protein antigen, and a carbohydrate antigen. The method includes (a) collecting, in a tube, fluid that potentially contains the particulate, using a plunger to push the fluid through a filter disposed at a distal portion of the tube or at a distal end of the plunger, and subsequently, while the filter is inside the tube, ascertaining if any of the particulate was trapped by the filter by applying a particulate-presence-testing-facilitation solution to the filter. Other embodiments are also described.

### SUMMARY OF THE APPLICATION

Diagnostic device efficacy is dependent on the accurate detection of an analyte present exclusively or at a known concentration range in samples procured from diseased subjects. However, it is common that the specificity of diagnostic devices is deleteriously affected by the presence of particulates found in samples procured from healthy subjects due to antibody cross-reactivity in immunological assays, or partial sequence homology in an amplicon of interest in molecular assays, resulting in false positive diagnostic outcomes. When possible, sample collection may be performed in a manner that avoids physical regions known to be rich in particulates which generate a false positive result, such as in the case of group A streptococcus identification, where there typically is a requirement for throat swab procurement to avoid contact with the tongue, cheeks, and teeth of a subject. However, specimen types such as oral rinses, saliva, and gargled liquid inherently sample large regions of the oral cavity, which may adversely affect their suitability for some diagnostic tests, particularly those that have low specificity.

Some applications of the invention described herein provide techniques to reduce and/or remove particulates which may generate a false positive diagnostic outcome from the oral cavity, making sampling of large regions of the oral cavity, such as oral rinse, saliva, and gargle specimens, as described above, more useful for diagnostic applications. In brief, the techniques of some applications of the present invention comprise performing an oral cavity rinsing prior to oral cavity sampling (typically by gargling), or alternatively, or in conjunction with, toothbrushing prior to oral cavity sampling.

These techniques may improve testing specificity by removing competing or cross-reacting bacteria from the oral cavity before gargling. (In applications in which the rinsing is performed without first brushing the teeth, the rinsing out the bacteria from the oral cavity does not provide any therapeutic benefit.) In addition, these techniques may improve test sensitivity by removing saliva and/or mucus from the oral cavity prior to gargling, thereby reducing the amount of saliva and/or mucus in the gargled liquid that is collected as the liquid specimen sample, without also materially reducing the concentration of the particulate of interest in the subsequently gargled fluid; as a result, the liquid specimen sample may be more easily filtered for subsequent testing for the presence of a particulate. Alternatively or additionally, these techniques (even rinsing alone) may very temporarily remove saliva (and thus acidity) from the oral cavity (without providing any therapeutic benefit) and thus improve the accuracy of molecular tests (e.g., isothermal tests or PCR tests) by removing acidity from the liquid specimen sample. Excess acidity sometimes causes errors in molecular tests.

Also provided in some applications of the present invention is a diagnostic kit containing the necessary components to facilitate the above techniques.

There is therefore provided, in accordance with an Inventive Concept 1 of the present invention, a method comprising:
rinsing an oral cavity of a subject with a rinse liquid;
thereafter, introducing into the oral cavity a gargle liquid and gargling the gargle liquid by the subject; and
thereafter, collecting at least a portion of the gargled liquid as a specimen sample.

Inventive Concept 2. The method according to Inventive Concept 1, wherein collecting the at least a portion of the gargled liquid as the specimen sample comprises spitting the at least a portion of the gargled liquid into a container.

Inventive Concept 3. The method according to Inventive Concept 1, wherein the rinse liquid is acidic.

Inventive Concept 4. The method according to Inventive Concept 1, wherein the rinse liquid has a neutral pH.

Inventive Concept 5. The method according to Inventive Concept 1, wherein the rinse liquid has a pH of 5-9.5.

Inventive Concept 6. The method according to Inventive Concept 5, wherein the rinse liquid has a pH of 5-7.

Inventive Concept 7. The method according to Inventive Concept 5, wherein the rinse liquid has a pH of 6-8.5.

Inventive Concept 8. The method according to Inventive Concept 5, wherein the rinse liquid has a pH of 7.1-9.5.

Inventive Concept 9. The method according to Inventive Concept 1, wherein the rinse liquid has a volume of 3-10 milliliters.

Inventive Concept 10. The method according to Inventive Concept 1, wherein the rinse liquid has a volume of 10-50 milliliters.

Inventive Concept 11. The method according to Inventive Concept 1, wherein the rinse liquid has a volume of 50-75 milliliters.

Inventive Concept 12. The method according to Inventive Concept 1, wherein rinsing comprises performing multiple successive rinses.

Inventive Concept 13. The method according to Inventive Concept 12, wherein the rinse liquid has a volume of more than 300 milliliters.

Inventive Concept 14. The method according to Inventive Concept 1, wherein rinsing the oral cavity with the rinse liquid comprises drawing the rinse liquid from a faucet connected to a public water supply.

Inventive Concept 15. The method according to Inventive Concept 1, wherein the rinse liquid is tap water.

Inventive Concept 16. The method according to Inventive Concept 1, wherein the gargle liquid is acidic.

Inventive Concept 17. The method according to Inventive Concept 1, wherein the gargle liquid has a neutral pH.

Inventive Concept 18. The method according to Inventive Concept 1, wherein the gargle liquid has a pH of 7.1-9.5.

Inventive Concept 19. The method according to Inventive Concept 1, wherein gargling the gargle liquid comprises drawing the gargle liquid from a faucet connected to a public water supply.

Inventive Concept 20. The method according to Inventive Concept 1, wherein the gargle liquid is tap water.

Inventive Concept 21. The method according to Inventive Concept 1, wherein the rinse liquid comprises at least 90% H2O by weight.

Inventive Concept 22. The method according to Inventive Concept 21, wherein the rinse liquid comprises at least 99% H2O by weight.

Inventive Concept 23. The method according to Inventive Concept 1, wherein the gargle liquid comprises at least 90% H2O by weight.

Inventive Concept 24. The method according to Inventive Concept 23, wherein the gargle liquid comprises at least 99% H2O by weight.

Inventive Concept 25. The method according to Inventive Concept 1, wherein the gargle liquid has a salt concentration of 0-0.154 mol/L.

Inventive Concept 26. The method according to Inventive Concept 1, wherein the gargle liquid has a salt concentration of 0.155-0.5 mol/L.

Inventive Concept 27. The method according to Inventive Concept 1, wherein the gargle liquid has a sweet taste.

Inventive Concept 28. The method according to Inventive Concept 27, wherein the gargle liquid has a salt concentration of 0-0.154 mol/L.

Inventive Concept 29. The method according to Inventive Concept 27, wherein the gargle liquid has a salt concentration of 0.155-0.5 mol/L.

Inventive Concept 30. The method according to Inventive Concept 1, wherein the gargle liquid is colored.

Inventive Concept 31. The method according to Inventive Concept 1, wherein the rinse liquid does not comprise a drug molecule.

Inventive Concept 32. The method according to Inventive Concept 1, wherein the gargle liquid does not comprise a drug molecule.

Inventive Concept 33. The method according to Inventive Concept 1, wherein the gargle liquid does not include a tastant.

Inventive Concept 34. The method according to Inventive Concept 1, wherein the gargle liquid has a salty taste or does not include a tastant.

Inventive Concept 35. The method according to Inventive Concept 1,
wherein the method further comprises, before gargling the gargle liquid, intranasally dispensing nasal wash fluid into a nasal cavity of the subject such that the nasal wash fluid washes biological material into an oropharynx of the subject from (a) the nasal cavity, (b) a nasopharynx of the subject, or (c) the nasal cavity and the nasopharynx,
wherein gargling the gargle liquid comprises:
   after intranasally dispensing the nasal wash fluid into the nasal cavity, orally dispensing the gargle liquid into the oral cavity via an anterior opening of the oral cavity; and
   gargling, by the subject, the gargle liquid, such that the gargle liquid forms a mixture with the nasal wash fluid, and
wherein collecting the at least a portion of the gargled liquid as the specimen sample comprises collecting, as the specimen sample, the mixture that passed out of the anterior opening of the oral cavity and contains at least a portion of the biological material washed into the oropharynx with the nasal wash fluid.

Inventive Concept 36. The method according to any one of Inventive Concepts 1-35, wherein the method does not comprise brushing teeth of the subject.

Inventive Concept 37. The method according to any one of Inventive Concepts 1-35, further comprising, before rinsing the oral cavity, cleaning teeth of the subject.

Inventive Concept 38. The method according to Inventive Concept 37, wherein cleaning the teeth comprises brushing the teeth, optionally brushing the teeth and gingiva of the subject.

Inventive Concept 39. The method according to Inventive Concept 38, wherein brushing the teeth comprises brushing the teeth using a toothbrush.

Inventive Concept 40. The method according to Inventive Concept 39, wherein the toothbrush has soft bristles.

Inventive Concept 41. The method according to Inventive Concept 40, wherein the soft bristles have a thickness of no more than 0.22 mm.

Inventive Concept 42. The method according to Inventive Concept 41, wherein the thickness is 0.1 mm-0.22 mm.

Inventive Concept 43. The method according to Inventive Concept 41, wherein the thickness is less than 0.1 mm.

Inventive Concept 44. The method according to Inventive Concept 39, wherein the toothbrush has medium or hard bristles.

Inventive Concept 45. The method according to Inventive Concept 38, wherein brushing the teeth comprises brushing the teeth using an oral swab.

Inventive Concept 46. The method according to Inventive Concept 38, wherein brushing the teeth comprises brushing the teeth with toothpaste.

Inventive Concept 47. The method according to Inventive Concept 46, wherein the toothpaste includes a coloring, and wherein rinsing comprises rinsing the oral cavity until the coloring is not visible in the spit-out rinse liquid by the naked human eye.

Inventive Concept 48. The method according to Inventive Concept 38, wherein brushing the teeth comprises brushing the teeth without toothpaste.

Inventive Concept 49. The method according to any one of Inventive Concepts 1-48, further comprising after collecting the specimen sample, testing the specimen sample for the presence of one or more particulates.

Inventive Concept 50. The method according to Inventive Concept 49, wherein testing the specimen sample for the presence of the one or more particulates comprises diagnosing a disease.

Inventive Concept 51. The method according to Inventive Concept 49, wherein testing the specimen sample for the presence of the one or more particulates comprises concentrating the specimen sample by filtration, by passing the specimen sample through one or more filters.

Inventive Concept 52. The method according to Inventive Concept 49, wherein testing the specimen sample for the presence of the one or more particulates comprises performing an immunoassay.

Inventive Concept 53. The method according to Inventive Concept 52, wherein performing the immunoassay comprises using a lateral flow immunoassay test strip.

Inventive Concept 54. The method according to Inventive Concept 53, wherein performing the immunoassay comprises using an image sensor and software to read and interpret the lateral flow immunoassay test strip.

Inventive Concept 55. The method according to Inventive Concept 49, wherein testing the specimen sample for the presence of the one or more particulates comprises using a molecular-based assay to test the specimen sample for the presence of the one or more particulates.

Inventive Concept 56. The method according to Inventive Concept 55, wherein testing the specimen sample for the presence of the one or more particulates comprises using nucleic acid amplification to test the specimen sample for the presence of the one or more particulates.

Inventive Concept 57. The method according to Inventive Concept 56, wherein testing the specimen sample for the presence of the one or more particulates comprises using polymerase chain reaction (PCR) to test the specimen sample for the presence of the one or more particulates.

Inventive Concept 58. The method according to Inventive Concept 56, wherein testing the specimen sample for the presence of the one or more particulates comprises using isothermal amplification to test the specimen sample for the presence of the one or more particulates.

Inventive Concept 59. The method according to Inventive Concept 49, wherein the one or more particulates are selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor.

Inventive Concept 60. The method according to Inventive Concept 49, wherein the one or more particulates are selected from the group consisting of: a virus, a bacterium, a protozoan, and a fungus.

Inventive Concept 61. The method according to Inventive Concept 60, wherein the one or more particulates include the bacterium.

Inventive Concept 62. The method according to Inventive Concept 61, wherein the bacterium is a Streptococcus bacterium.

Inventive Concept 63. The method according to Inventive Concept 60, wherein the one or more particulates include a virus.

Inventive Concept 64. The method according to Inventive Concept 63, wherein the virus is an Influenza virus.

Inventive Concept 65. The method according to Inventive Concept 63, wherein the virus is Epstein-Barr virus.

Inventive Concept 66. The method according to Inventive Concept 63, wherein the virus is respiratory syncytial virus (RSV).

Inventive Concept 67. The method according to Inventive Concept 63, wherein the virus is a coronavirus.

Inventive Concept 68. The method according to Inventive Concept 67, wherein the coronavirus is Severe acute respiratory syndrome coronavirus 2 (SARS-CoV-2).

Inventive Concept 69. The method according to Inventive Concept 60, wherein the one or more particulates include one or more antigens selected from the group consisting of: a viral antigen, a bacterial antigen, and a fungal antigen.

There is further provided, in accordance with an Inventive Concept 70 of the present invention, a test kit for performing a test on a subject, the test kit comprising:
a rinse-liquid container containing a rinse liquid;
a gargle-liquid container containing a gargle liquid, for gargling by the subject to produce a specimen sample including at least a portion of the gargled liquid;
a test configured to detect the presence of one or more particulates in the specimen sample; and
packaging, in which the rinse-liquid container, the gargle-liquid container, and the test are removably packaged.

Inventive Concept 71. The test kit according to Inventive Concept 70, further comprising a container for collecting the specimen sample for use with the test.

Inventive Concept 72. The test kit according to Inventive Concept 70, wherein the gargle liquid is acidic.

Inventive Concept 73. The test kit according to Inventive Concept 70, wherein the gargle liquid has a neutral pH.

Inventive Concept 74. The test kit according to Inventive Concept 70, wherein the gargle liquid has a pH of 7.1-9.5.

Inventive Concept 75. The test kit according to Inventive Concept 70, wherein the gargle liquid comprises at least 90% H2O by weight.

Inventive Concept 76. The test kit according to Inventive Concept 75, wherein the gargle liquid comprises at least 99% H2O by weight.

Inventive Concept 77. The test kit according to Inventive Concept 70, wherein the gargle liquid has a salt concentration of 0-0.154 mol/L.

Inventive Concept 78. The test kit according to Inventive Concept 70, wherein the gargle liquid has a salt concentration of 0.155-0.5 mol/L.

Inventive Concept 79. The test kit according to Inventive Concept 70, wherein the gargle liquid has a sweet taste.

Inventive Concept 80. The test kit according to Inventive Concept 79, wherein the gargle liquid has a salt concentration of 0-0.154 mol/L.

Inventive Concept 81. The test kit according to Inventive Concept 79, wherein the gargle liquid has a salt concentration of 0.155-0.5 mol/L.

Inventive Concept 82. The test kit according to Inventive Concept 70, wherein the gargle liquid is colored.

Inventive Concept 83. The test kit according to Inventive Concept 70, wherein the gargle liquid does not comprise a drug molecule.

Inventive Concept 84. The test kit according to Inventive Concept 70, wherein the gargle liquid does not include a tastant.

Inventive Concept 85. The test kit according to Inventive Concept 70, wherein the gargle liquid has a salty taste or does not include a tastant.

Inventive Concept 86. The test kit according to Inventive Concept 70, further comprising:
a nasal wash fluid; and
a nasal wash fluid dispenser, which contains the nasal wash fluid and is configured to intranasally dispense the nasal wash fluid into a nasal cavity of the subject.

Inventive Concept 87. The test kit according to any one of Inventive Concepts 70-86, further comprising a dental cleaner selected from the group consisting of: a toothbrush and an oral swab.

Inventive Concept 88. The test kit according to Inventive Concept 87, wherein the dental cleaner comprises the toothbrush, and wherein the toothbrush comprises soft bristles.

Inventive Concept 89. The test kit according to Inventive Concept 88, wherein the bristles have a thickness of no more than 0.22 mm.

Inventive Concept 90. The test kit according to Inventive Concept 89, wherein the thickness is 0.1 mm-0.22 mm.

Inventive Concept 91. The test kit according to Inventive Concept 89, wherein the thickness is less than 0.1 mm.

Inventive Concept 92. The test kit according to Inventive Concept 87, wherein the toothbrush has medium or hard bristles.

Inventive Concept 93. The test kit according to Inventive Concept 87, wherein the dental cleaner comprises the oral swab.

Inventive Concept 94. The test kit according to any one of Inventive Concepts 70-93, wherein the test kit further comprises a toothpaste container containing toothpaste, the toothpaste container removably packaged in the packaging.

Inventive Concept 95. The test kit according to Inventive Concept 94, wherein the toothpaste comprises a coloring.

Inventive Concept 96. The test kit according to any one of Inventive Concepts 70-95, wherein the test kit further comprises one or more filters for filtering the specimen sample.

Inventive Concept 97. The test kit according to any one of Inventive Concepts 70-86, wherein the test comprises a lateral flow immunoassay test strip.

Inventive Concept 98. The test kit according to any one of Inventive Concepts 70-96, wherein the test comprises an immunoassay.

Inventive Concept 99. The test kit according to Inventive Concept 98, wherein the immunoassay comprises a lateral flow immunoassay test strip.

Inventive Concept 100. The test kit according to any one of Inventive Concepts 70-96, wherein the test comprises a molecular-based assay.

Inventive Concept 101. The test kit according to any one of Inventive Concepts 70-100, wherein the one or more particulates are selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor, and wherein the test is configured to detect the presence of the one or more particulates.

Inventive Concept 102. The test kit according to any one of Inventive Concepts 70-100, wherein the one or more particulates are selected from the group consisting of: a virus, a bacterium, a protozoan, and a fungus.

Inventive Concept 103. The test kit according to Inventive Concept 102, wherein the one or more particulates include the bacterium.

Inventive Concept 104. The test kit according to Inventive Concept 103, wherein the bacterium is a Streptococcus bacterium.

Inventive Concept 105. The test kit according to Inventive Concept 102, wherein the one or more particulates include a virus.

Inventive Concept 106. The test kit according to Inventive Concept 105, wherein the virus is an Influenza virus.

Inventive Concept 107. The test kit according to Inventive Concept 105, wherein the virus is Epstein-Barr virus.

Inventive Concept 108. The apparatus according to Inventive Concept 105, wherein the virus is respiratory syncytial virus (RSV).

Inventive Concept 109. The test kit according to Inventive Concept 105, wherein the virus is a coronavirus.

Inventive Concept 110. The test kit according to Inventive Concept 109, wherein the coronavirus is Severe acute respiratory syndrome coronavirus 2 (SARS-CoV-2).

Inventive Concept 111. The test kit according to Inventive Concept 102, wherein the one or more particulates includes one or more antigens selected from the group consisting of: a viral antigen, a bacterial antigen, and a fungal antigen.

Inventive Concept 112. The test kit according to any one of Inventive Concepts 70-96, wherein the rinse liquid has a pH of 5-9.5.

Inventive Concept 113. The test kit according to Inventive Concept 112, wherein the rinse liquid has a pH of 5-7.

Inventive Concept 114. The test kit according to Inventive Concept 112, wherein the rinse liquid has a pH of 6-8.5.

There is still further provided, in accordance with an Inventive Concept 115 of the present invention, a method comprising:
rinsing an oral cavity of a subject with a rinse liquid;
thereafter, spitting out, by the subject, the rinse liquid;
thereafter waiting a period of time for fresh saliva to build up in the oral cavity;
thereafter, spitting, by the subject, a specimen sample out of the oral cavity; and
thereafter, collecting at least a portion of the spit specimen sample.

Inventive Concept 116. The method according to Inventive Concept 115, wherein the waiting time between spitting out the rinse liquid and spitting out the specimen sample is less than 2 hours.

Inventive Concept 117. The method according to Inventive Concept 116, wherein the waiting time between spitting out the rinse liquid and spitting out the specimen sample is less than 1 hour.

Inventive Concept 118. The method according to Inventive Concept 117, wherein the waiting time between spitting out the rinse liquid and spitting out the specimen sample is less than 15 minutes.

Inventive Concept 119. The method according to Inventive Concept 118, wherein the waiting time between spitting out the rinse liquid and spitting out the specimen sample is less than 5 minutes.

Inventive Concept 120. The method according to Inventive Concept 119, wherein the waiting time between spitting out the rinse liquid and spitting out the specimen sample is less than 30 seconds.

Inventive Concept 121. The method according to any one of Inventive Concepts 115-120, further comprising, before rinsing the oral cavity, cleaning teeth of the subject.

Inventive Concept 122. The method according to Inventive Concept 121, wherein cleaning the teeth comprises brushing the teeth.

Inventive Concept 123. The method according to Inventive Concept 122, wherein brushing the teeth comprises brushing the teeth using a toothbrush.

Inventive Concept 124. The method according to Inventive Concept 122, wherein brushing the teeth comprises brushing the teeth with toothpaste.

Inventive Concept 125. The method according to any one of Inventive Concepts 115-124, further comprising after collecting the specimen sample, testing the specimen sample for the presence of one or more particulates.

Inventive Concept 126. The method according to Inventive Concept 125, wherein testing the specimen sample for the presence of the one or more particulates comprises diagnosing a disease.

Inventive Concept 127. The method according to Inventive Concept 51, wherein concentrating the specimen sample by filtration comprises passing the specimen sample through the one or more filters within 60 minutes of finishing collecting the at least a portion of the gargled liquid as the specimen sample.

Inventive Concept 128. The method according to any one of Inventive Concepts 1-69 and 127, wherein introducing the gargle liquid into the oral cavity comprises introducing the gargle liquid into the oral cavity within 5 minutes of finishing rinsing the oral cavity.

The present invention will be more fully understood from the following detailed description of embodiments thereof, taken together with the drawings, in which:

### BRIEF DESCRIPTION OF THE DRAWINGS

FIGS. 1A-D are schematic illustration of respective configurations of a test kit, in accordance with respective applications of the present invention;

FIG. 2 is a flowchart schematically illustrating a method, in accordance with an application of the present invention;

FIG. 3 is a flowchart schematically illustrating another method, in accordance with an application of the present invention;

FIGS. 4A-D are schematic illustrations of a sampling device for concentrating a liquid specimen sample and a method of using the sampling device, in accordance with an application of the present invention; and

FIGS. 5A-D are schematic cross-sectional illustrations of the sampling device of FIGS. 4A-B and the method of using the sampling device, in accordance with an application of the present invention.

### DETAILED DESCRIPTION OF APPLICATIONS

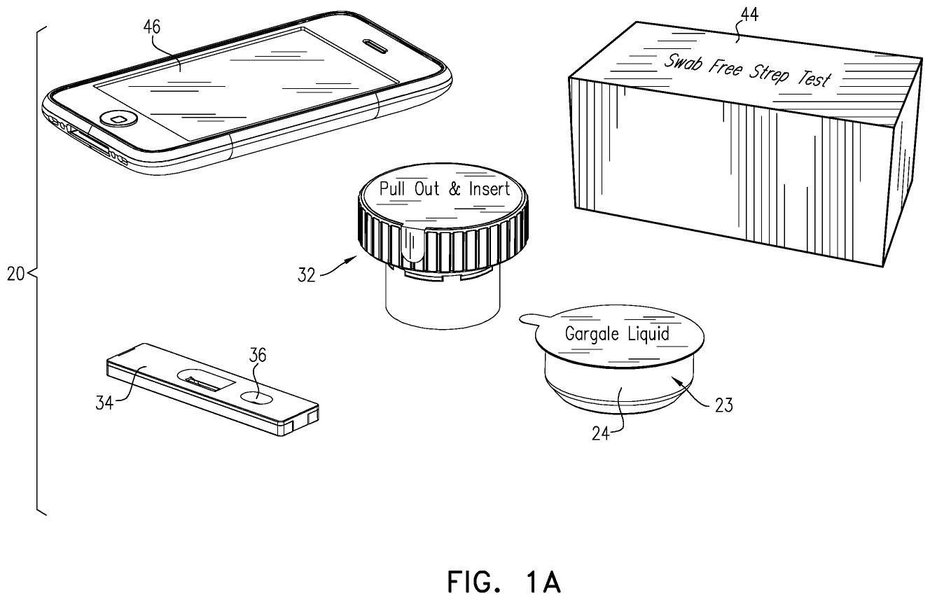

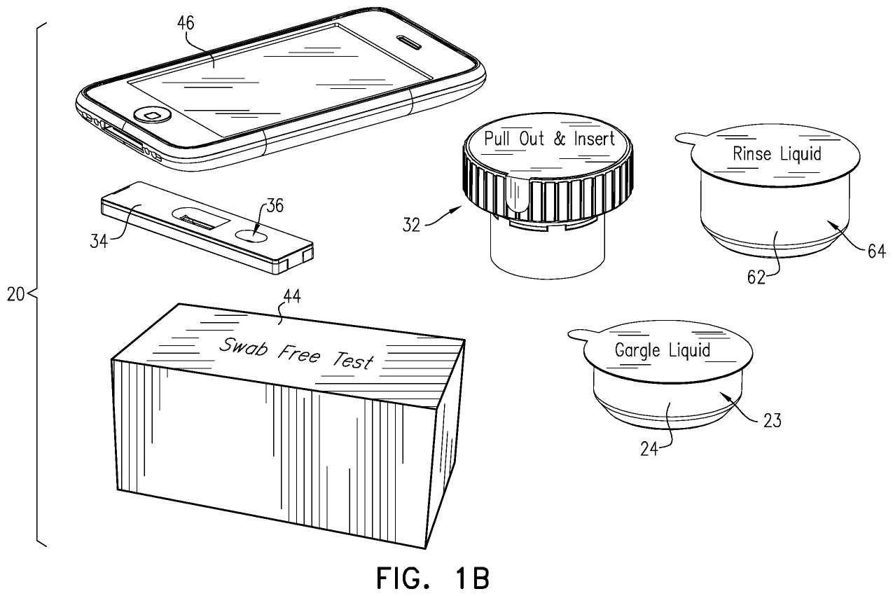

FIGS. 1A-D are schematic illustration of respective configurations of a test kit in accordance with respective applications of the present invention. Typically, test kit 20 comprises a gargle liquid 23, for gargling by a subject to produce a specimen sample comprising at least a portion of the gargled liquid. Gargle liquid 23 is typically contained in a gargle-liquid container 24.

Typically, test kit 20 further comprises a test 34 (e.g., a diagnostic test), which is configured to detect the presence of one or more particulates (such as those described herein) in the specimen sample. Test 34 may optionally be configured to implement testing techniques described herein, in the patent applications and publications incorporated hereinbelow by reference, and/or known in the art. For some applications, test 34 comprises a lateral flow test strip 36 (e.g., a lateral flow immunoassay test strip), which is configured to detect the presence of the particulate (such as by detecting protein antigen, e.g., from a bacterium or a virus), and, optionally, one or more reagents. Alternatively, for some applications, test 34 comprises a rapid molecular test kit, for example one that uses a real-time loop mediated amplification reaction. Further alternatively, test 34 may comprise a CRISPR-based diagnostic test, an ELISA diagnostic test, or a spectroscopy-based diagnostic test.

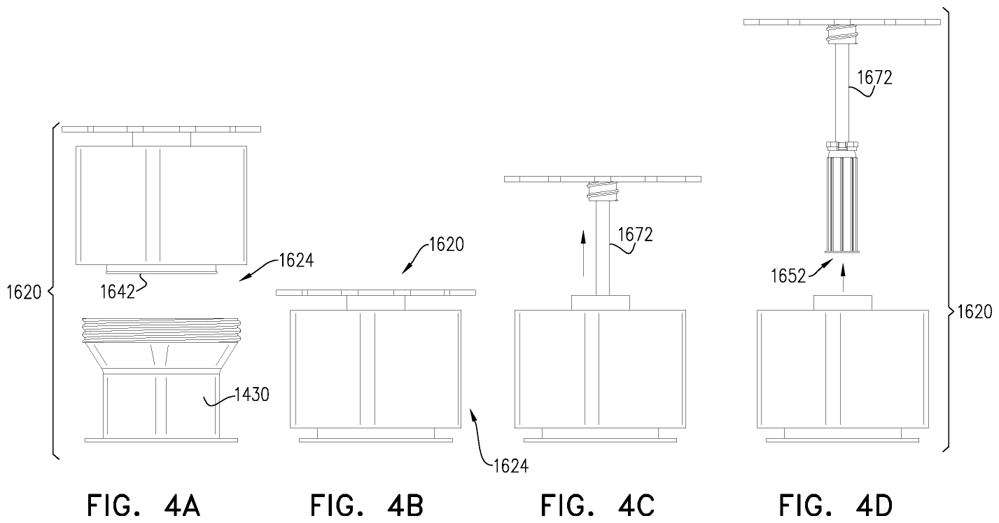

For some applications, test kit 20 may comprise one or more filters for filtering the specimen sample. Optionally, test 34 and/or the one or more filters may implement any of the filtering techniques described in one or more of the patent applications and publications incorporated hereinbelow by reference. For some of these applications, test kit 20 further comprises a filter unit 32 that comprises the one or more filters. Filter unit 32 may implement any of the techniques of the filter units described in the patent applications and publications incorporated hereinbelow by reference; by way of example and not limitation, filter unit 32 may (a) comprise sampling device 1620, described hereinbelow with reference to FIGS. 4A-D and 5A-D, and/or implement any of the techniques of sampling device 1620, (b) implement the techniques of any of the sampling devices described in WO 2022/149135 to Feldman et al., and/or (c) may implement the techniques of one of filter units 500A or 500B, described in PCT Publication WO 2021/224925 to Levitz et al. with reference to FIGS. 14A-B, respectively.

In configurations in which test kit 20 further comprises filter unit 32, test 34 is configured to test for the presence of a particulate (such as those described herein) trapped by the one or more filters of filter unit 32.

For some applications, test kit 20 further comprises a container for collecting the specimen sample for use with test 34. Optionally, the container is an element of filter unit 32.

Reference is made to FIG. 1B. For some applications, test kit 20 further comprises a rinse liquid 64, for rinsing an oral cavity of the subject. Rinse liquid 64 is typically contained in a rinse-liquid container 62. For some applications, rinse liquid 64 has a volume of 3-10 milliliters, 10-50 milliliters, or 50-75 milliliters.

Reference is made to FIGS. 1C-D. For some applications, test kit 20 further comprises a dental cleaner 26, which typically comprises a toothbrush 28 (such as shown in FIG. 1C) or an oral swab 30 (such as shown in FIG. 1D). Toothbrush 28 is optionally a conventional toothbrush, such as shown. Oral swab 30 is optionally a conventional oral swab, such as shown. An oral swab may also be known as a dental sponge swabstick, and comprises a handle 50 and a sponge 52, e.g., a synthetic sponge or a luffa sponge.

For some applications, test kit 20 further comprises a toothpaste container 40 containing toothpaste 42.

Reference is made to FIGS. 1A-D. Typically, test kit 20 further comprises packaging 44, in which some or all of the elements of test kit 20 are removably packaged. For example, one or more of toothpaste container 40, dental cleaner 26, gargle-liquid container 24, and test 34 may be removably packaged in packaging 44.

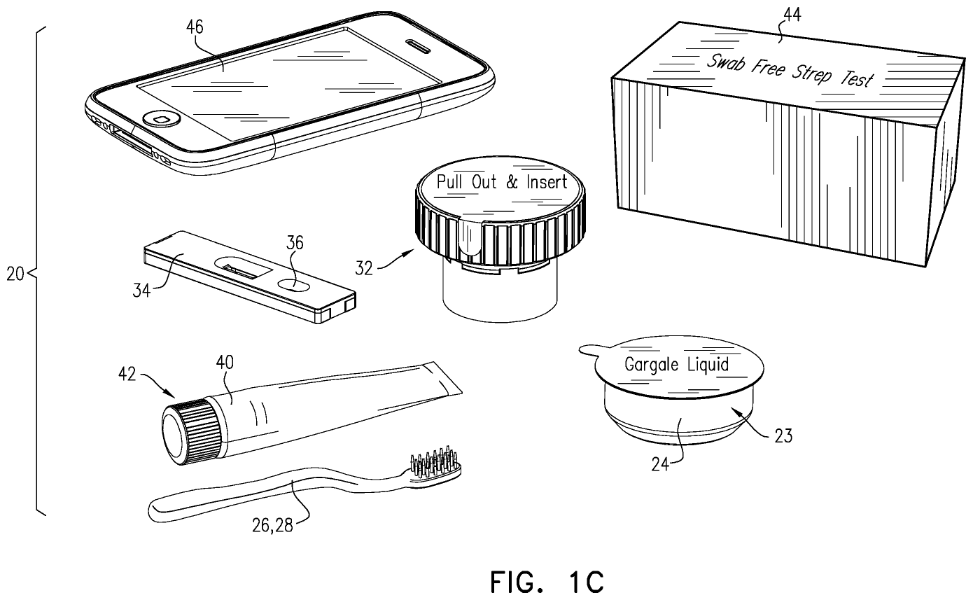

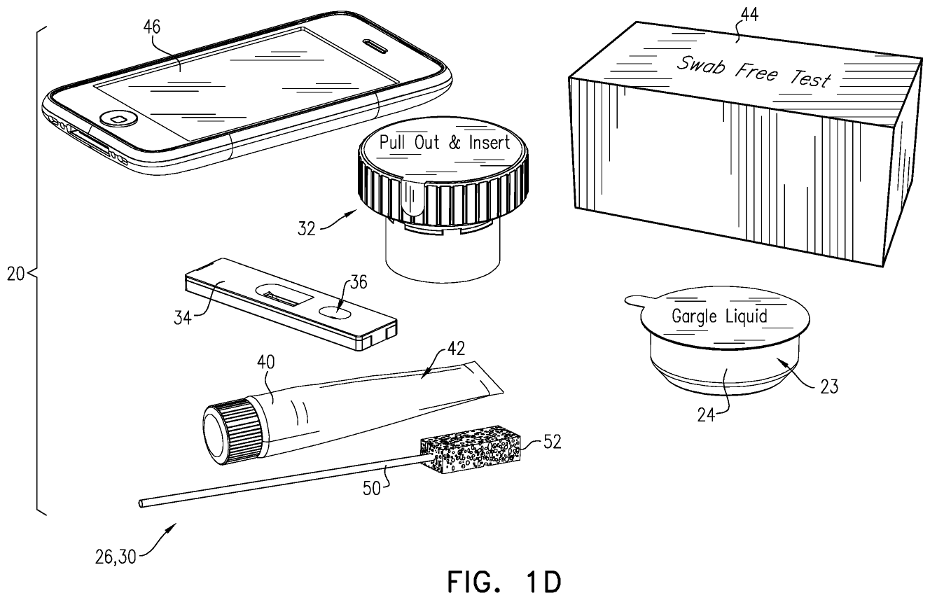

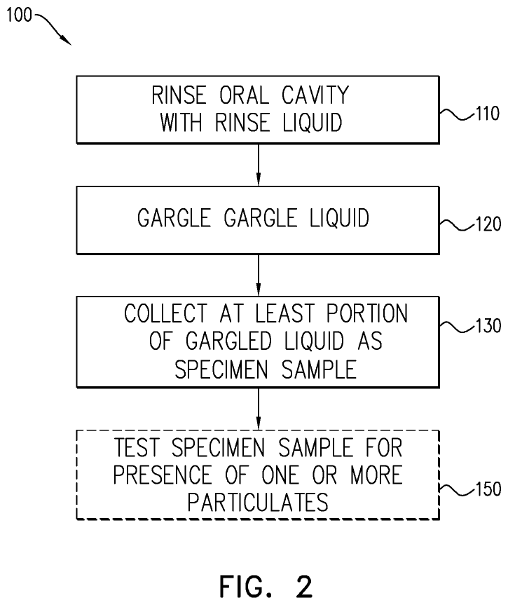

Reference is still made to FIGS. 1A-D, and is additionally made to FIG. 2, which is a flowchart schematically illustrating a method 100, in accordance with an application of the present invention. Method 100 is optionally performed using test kit or a portion of the elements of test kit 20. Alternatively, method 100 is performed without using test kit 20. Typically, method 100 is a non-therapeutic method.

Method 100 comprises:
at a rinse step 110, rinsing an oral cavity of a subject with rinse liquid 64 (including spitting out rinse liquid 64);
thereafter, at a gargling step 120, introducing into the oral cavity a gargle liquid 23 and gargling gargle liquid 23 by the subject; and
thereafter, at a collection step 130, collecting at least a portion of the gargled liquid as a specimen sample.

Gargling at gargling step 120 comprises introducing gargle liquid 23 into the oral cavity, i.e., orally dispensing gargle liquid 23 into the oral cavity via an anterior opening of the oral cavity. Gargle liquid 23 is thus separate and distinct from rinse liquid 64 (although they optionally may have the same composition, e.g., saline solution or water, e.g., tap water). Optionally, gargling gargle liquid 23 comprises gargling gargle liquid 23 that was contained in gargle-liquid container 24. As used in the present application, including in the claims and the Inventive Concepts, “gargling” means holding a liquid in the throat (typically in the oropharynx) and agitating with air expelled from the lungs. In general, during gargling, the subject avoids swallowing to the extent reasonably possible. In the present application, including in the claims and the Inventive Concepts, a “gargle liquid” refers to a liquid for gargling before the liquid has been gargled, and “gargled liquid” refers to the gargled liquid after it has been gargled.

For some applications, collecting the at least a portion of the gargled liquid as the specimen sample at collection step 130 comprises spitting the at least a portion of the gargled liquid, such as into a container.

Typically, the rinsing at rinse step 110 is performed for a minimum period of time. For example, the minimum period of time may be 3-15 seconds, such as 5-10 seconds. Alternatively or additionally, the rinsing at rinse step 110 is performed for at least 5 seconds, such as at least 10 seconds.

Typically, the rinsing at rinse step 110 comprises rinsing the teeth and the gingiva, including in regions between the teeth and the cheek and between the teeth and the inner surfaces of the lips. Optionally, the rinsing at rinse step 110 is performed by swishing rinse liquid 64 in the oral cavity.

Typically, rinsing the oral cavity at rinse step 110 of method 100 is non-therapeutic.

Typically, gargling gargle liquid 23 at gargling step 120 of method 100 is non-therapeutic.

Reference is still made to FIGS. 1A-D and 2. For some applications, rinse liquid 64 is acidic, e.g., has a pH of 1-3, 3-5, 5-6, or 6-7. For other applications, rinse liquid 64 has a neutral pH. For still other applications, rinse liquid 64 has a pH of 5-9.5, e.g., 6-9.5, such as 7.1-9.5, or 5-8.5, e.g., 5-7 or 6-8.5.

For some applications, rinsing at rinse step 110 comprises performing multiple successive rinses. For some of these applications, rinse liquid 64 has a volume of more than 300 milliliters, e.g., more than 360 milliliters, such as more than 400 milliliters.

For some applications, rinsing the oral cavity with rinse liquid 64 at rinse step 110 comprises drawing rinse liquid 64 from a faucet connected to a public water supply. For some applications, rinse liquid 64 is tap water.

For some applications, rinse liquid 64 comprises at least 90% H2O by weight, such as at least 99% H2O by weight.

For some applications, rinse liquid 64 does not comprise a drug molecule. As used in the present application, including in the claims and the Inventive Concepts, a drug molecule means a chemical substance used in the treatment, cure, prevention, or diagnosis of disease.

For some applications, gargle liquid 23 is acidic, e.g., has a pH of 1-3, 3-5, 5-6, or 6-7. For other applications, gargle liquid 23 has a neutral pH. For still other applications, gargle liquid 23 has a pH of 7.1-9.5.

For some applications, introducing gargle liquid 23 into the oral cavity at gargling step 120 comprises introducing gargle liquid 23 into the oral cavity within 5 minutes of finishing rinsing the oral cavity at rinse step 110, such as within two minutes, e.g., within 60 seconds, such as within 30 seconds, e.g., within 10 seconds. A shorter period of time between rinsing and gargling generally reduces the amount of saliva that reaccumulates in the oral cavity after rinsing.

For some applications, gargling gargle liquid 23 comprises drawing gargle liquid 23 from a faucet connected to a public water supply. For some applications, gargle liquid 23 is tap water.

For some applications, gargle liquid 23 has a volume of 5-10 milliliters.

For some applications, gargle liquid 23 comprises at least 90% H2O by weight, such as at least 99% H2O by weight.

For some applications, gargle liquid 23 has a salt concentration of 0-0.154 mol/L or 0.155-0.5 mol/L. For example, the saline solution may be hypertonic, isotonic, or hypotonic (for example, a phosphate-buffered saline solution).

For some applications, gargle liquid 23 has a sweet taste. For example, gargle liquid 23 may comprise a natural or artificial sweetener, a natural or artificial flavor, and/or a scent. For some of these applications, gargle liquid 23 has a salt concentration of 0-0.154 mol/L, or a salt concentration of 0.155-0.5 mol/L. Typically, the saltiness of gargle liquid 23 is at least partially concealed by the sweet taste of gargle liquid 23.

For some applications, gargle liquid 23 comprises a tastant, for example, menthol flavoring. For other applications, gargle liquid 23 does not comprise a tastant. For some applications, gargle liquid 23 has a salty taste or does not comprise a tastant.

Typically, gargle liquid 23 is not colored (coloring may interfere with the reading of the test line and/or control line on a lateral flow test strip). Alternatively, gargle liquid 23 is colored.

For some applications, gargle liquid 23 does not comprise a drug molecule.

Reference is made to FIG. 2. For some applications:
method 100 further comprises, before gargling gargle liquid 23, intranasally dispensing nasal wash fluid into a nasal cavity of the subject such that the nasal wash fluid washes biological material into an oropharynx of the subject from (a) the nasal cavity, (b) a nasopharynx of the subject, or (c) the nasal cavity and the nasopharynx. This portion of method 100 may be performed using techniques described in PCT Publication WO 2021/224925 to Levitz et al., U.S. application Ser. No. 17/980,200, filed Nov. 3, 2022, and/or any of the other patent applications incorporated herein by reference hereinbelow; all of these applications are assigned to the assignee of the present application;
gargling the gargle liquid comprises (a) after intranasally dispensing the nasal wash fluid into the nasal cavity, orally dispensing gargle liquid 23 into the oral cavity via an anterior opening of the oral cavity, and (b) gargling, by the subject, gargle liquid 23, such that gargle liquid 23 forms a mixture with the nasal wash fluid; and
collecting the at least a portion of the gargled liquid as the specimen sample comprises collecting, as the specimen sample, the mixture that passed out of the anterior opening of the oral cavity and contains at least a portion of the biological material washed into the oropharynx with the nasal wash fluid.

Reference is made to FIG. 1A-D. For some applications, test kit 20 further comprises a nasal wash fluid; and a nasal wash fluid dispenser, which contains the nasal wash fluid and is configured to intranasally dispense the nasal wash fluid into a nasal cavity of the subject. The nasal wash fluid may implement any of the techniques described in PCT Publication WO 2021/224925 to Levitz et al., U.S. application Ser. No. 17/980,200, filed Nov. 3, 2022, and/or any of the other patent applications incorporated herein by reference hereinbelow. The nasal wash fluid dispenser may be conventional, or may implement any of the techniques described in PCT Publication WO 2021/224925 to Levitz et al., U.S. application Ser. No. 17/980,200, filed Nov. 3, 2022, and/or any of the other patent applications incorporated herein by reference hereinbelow.

Reference is still made to FIG. 2. For any of the applications described hereinabove, method 100 may further comprise, before rinsing the oral cavity at rinse step 110, brushing (or otherwise cleaning) teeth of the subject. For some applications, brushing the teeth comprises brushing the teeth and gingiva of the subject. For some applications, brushing the teeth comprises brushing the teeth using toothbrush 28.

Reference is again made to FIGS. 1A-D and 2. For some applications, toothbrush 28 has soft bristles. For some applications, the soft bristles have a thickness of no more than 0.22 mm. For some applications, the thickness is 0.1 mm-0.22 mm. For some applications, the thickness is less than 0.1 mm. Alternatively, for some applications, toothbrush 28 has medium or hard bristles.

Reference is made to FIG. 2. For some applications, brushing the teeth comprises brushing the teeth using oral swab 30. For some applications, brushing the teeth comprises brushing the teeth with toothpaste 42. For other applications, brushing the teeth comprises brushing the teeth without toothpaste. In applications in which the subject brushes the teeth (with or without toothpaste), a better specimen sample is generally obtained if the subject subsequently rinses the oral cavity well at rinse step 110.

In experiments conducted by the inventor, he observed that brushing the teeth, with or without toothpaste 42, results in greater contamination of the specimen sample if the subject does not subsequently rinse well, than if the specimen sample is collected after rinsing without brushing.

In some applications of the present invention, method 100 does not comprise brushing the teeth. Refraining from brushing the teeth may avoid possible contamination of the specimen sample if the subject does not rinse well after brushing the teeth.

For some applications in which brushing the teeth comprises brushing the teeth with toothpaste 42, toothpaste 42 comprises a coloring, and rinsing at rinse step 110 comprises rinsing the oral cavity until the coloring is not visible in the spit-out rinse liquid 64 by the naked human eye; this may help ensure the subject rinses well. Typically, the coloring is a non-clear coupling. Optionally, the coloring is a non-white coloring.

Reference is still made to FIG. 2. For any of the applications described hereinabove, method 100 may further comprise after collecting the specimen sample, testing the specimen sample for the presence of one or more particulates, at a testing step 150.

For some applications, testing the specimen sample for the presence of the one or more particulates at testing step 150 comprises diagnosing a disease.

For some applications, testing the specimen sample for the presence of the one or more particulates at testing step 150 comprises concentrating the specimen sample by filtration, by passing the specimen sample through one or more filters. This filtering may implement any of the filtering techniques described hereinbelow with reference to FIGS. 4A-D and 5A-D and/or in one or more of the patent applications and publications incorporated hereinbelow by reference.

For some applications, concentrating the specimen sample by filtration comprises passing the specimen sample through the one or more filters within 60 minutes of finishing collecting the at least a portion of the gargled liquid as the specimen sample, such as within 30 minutes, e.g., within 5 minutes, such as within 2 minutes. Filtering the specimen sample as soon as possible after collection may optimize size filtration of the one or more particulates before the one or more particulates have time to degrade to smaller components.

For some applications, testing the specimen sample for the presence of the one or more particulates at testing step 150 comprises performing an immunoassay, such as using a lateral flow immunoassay test strip.

For some applications, performing the immunoassay comprises using an image sensor and software to read and interpret the lateral flow immunoassay test strip. For example, the image sensor may be an element of a camera of a smartphone 46 (such as shown in FIG. 1A-D) or of another camera.

For some applications, testing the specimen sample for the presence of the one or more particulates at testing step 150 comprises using:
a molecular-based assay to test the specimen sample for the presence of the one or more particulates,
nucleic acid amplification to test the specimen sample for the presence of the one or more particulates,
polymerase chain reaction (PCR) to test the specimen sample for the presence of the one or more particulates, and/or
isothermal amplification to test the specimen sample for the presence of the one or more particulates.

Typically, the one or more particulates are one or more biological particulates.

For some applications, the one or more particulates are selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor. If provided, test 34 is configured to detect the presence of the one or more particulates.

As used in the present application, including in the claims and the Inventive Concepts, microorganisms may include either pathogenic microorganisms or non-pathogenic microorganisms or both, for example, viruses, bacteria, protozoa, and fungi.

For some applications, the one or more particulates are selected from the group consisting of: a virus, a bacterium, a protozoan, and a fungus.

For some applications, the one or more particulates comprise the bacterium. For some applications, the bacterium is a Streptococcus bacterium.

For some applications, the one or more particulates comprise a virus. For some applications, the virus is an Influenza virus. For some applications, the virus is Epstein-Barr virus. For some applications, the virus is respiratory syncytial virus (RSV). For some applications, the virus is a coronavirus. For some applications, the coronavirus is Severe acute respiratory syndrome coronavirus 2 (SARS-CoV-2).

For some applications, the one or more particulates comprise one or more antigens selected from the group consisting of: a viral antigen, a bacterial antigen, and a fungal antigen.

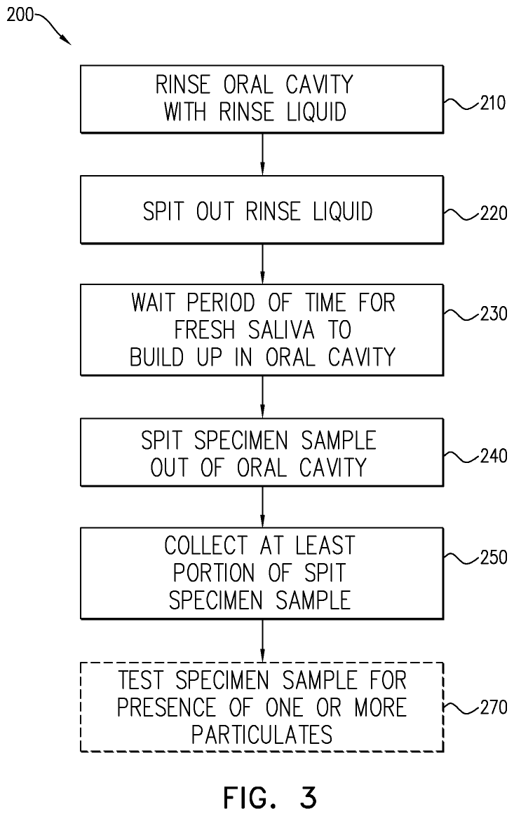

Reference is now made to FIG. 3, which is a flowchart schematically illustrating a method 200, in accordance with an application of the present invention. Method 200 comprises:
at a rinse step 210, rinsing an oral cavity of a subject with a rinse liquid;
thereafter, at a rinse spitting step 220, spitting out, by the subject, the rinse liquid;
thereafter, at a waiting step 230, waiting a period of time for fresh saliva to build up in the oral cavity;
thereafter, at a sample spitting step 240, spitting, by the subject, a specimen sample out of the oral cavity; and
thereafter, at a collection step 250, collecting at least a portion of the spit specimen sample.

For some applications, the waiting time between spitting out the rinse liquid and spitting out the specimen sample is less than 2 hours, such as less than 1 hour, e.g., less than 15 minutes, such as less than 5 minutes, e.g., less than 30 seconds.

Typically, the waiting time between spitting out the rinse liquid and spitting out the specimen sample is at least 15 seconds.

For any of the applications described hereinabove, method 200 may further comprise, before rinsing the oral cavity at rinse step 210, brushing (or otherwise cleaning) teeth of the subject. For some applications, brushing the teeth comprises brushing the teeth using a toothbrush. For some applications, brushing the teeth comprises brushing the teeth with toothpaste. For some applications, brushing the teeth comprises brushing the teeth using oral swab 30. The toothbrush, toothpaste, oral swab, and/or brushing may optionally implement any of the techniques of the toothbrush, toothpaste, oral swab, and/or brushing, respectively, described hereinabove with reference to FIGS. 1A-D and 2.

For any of the applications described hereinabove, method 200 may further comprise after collecting the specimen sample, testing the specimen sample for the presence of one or more particulates, at a testing step 270. Testing step 270 may optionally implement any of the techniques of testing step 150, described hereinabove with reference to FIGS. 1A-D and 2.

In some applications of the present invention, method 200 further comprises any of the steps of method 100, described hereinabove with reference to FIG. 2, mutatis mutandis .

Reference is now made to FIGS. 4A-D, which are schematic illustrations of a sampling device 1620 for concentrating a liquid specimen sample 22 and a method of using sampling device 1620, in accordance with an application of the present invention.

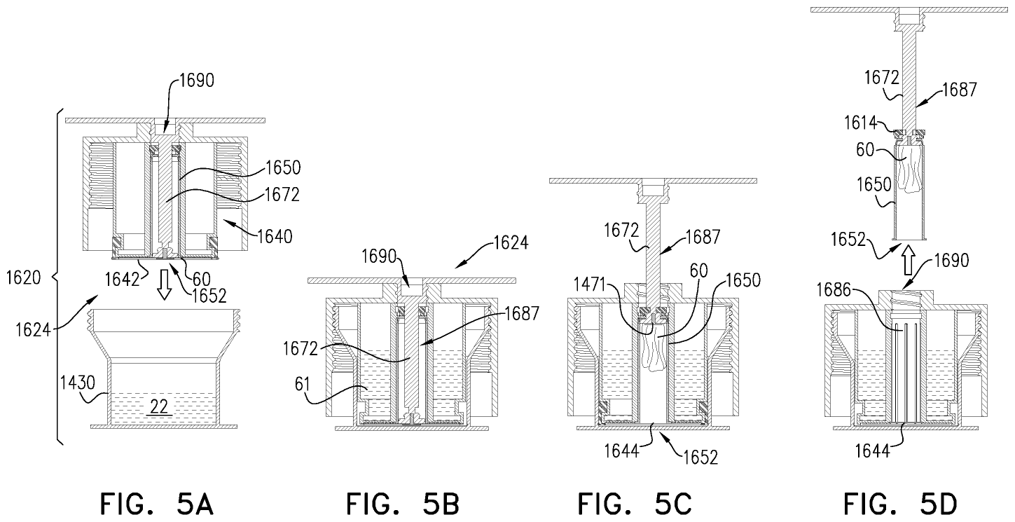

Reference is also made to FIGS. 5A-D, which are schematic cross-sectional illustrations of sampling device 1620 and the method of using sampling device 1620, in accordance with an application of the present invention.

Sampling device 1620 may implement any techniques of sampling device 1620 described in PCT Publication WO 2022/149135 to Feldman et al.

Sampling device 1620 comprises a filtration assembly 1624 and a collection vial 1650. Filtration assembly 1624 comprises a tubular container 1430, a plunger 1640 (labeled in FIG. 5A), and a filter 60. Filter 60 is configured to concentrate at least a portion of liquid specimen sample 22 onto filter 60, while allowing a filtrate 61 to pass through filter 60. Typically, distal advancement of plunger 1640 within tubular container 1430 applies pressure to drive (e.g., push) at least a portion of liquid specimen sample 22 contained in tubular container 1430 through filter 60, such as shown in the transitions between FIGS. 4A and 4B and between FIGS. 5A and 5B.

Sampling device 1620 is typically configured such that filter 60 is removable from tubular container 1430 via a plunger-space proximal opening 1690 while a plunger head 1642 of plunger 1640 is within tubular container 1430 (filter 60 is also removable from tubular container 1430 via plunger-space proximal opening 1690 if plunger head 1642 has been removed from tubular container 1430). Plunger head 1642 is shaped so as to define a plunger-head opening 1644 through plunger head 1642 and into an internal plunger space 1686 of plunger 1640.

Sampling device 1620 is configured such that filter 60 is advanceable into (e.g., entirely into) collection vial 1650 via a vial opening 1652 while collection vial 1650 is disengageably coupled to filtration assembly 1624, such as shown in FIGS. 4C and 5C.

For some applications, sampling device 1620 comprises a filter-withdrawal shaft 1672, which:
is disposed partially within collection vial 1650 within an internal plunger space 1686,
includes a proximal portion 1687 that is slidably disposed passing through a shaft-passage hole through an end of collection vial 1650 opposite vial opening 1652, and
includes a distal portion that is directly or indirectly coupled to filter 60.

Sampling device 1620 is configured such that proximal withdrawal of filter-withdrawal shaft 1672, while plunger head 1642 is within tubular container 1430, pulls filter 60 into collection vial 1650 via plunger-head opening 1644 via vial opening 1652 (as shown in the transitions between FIGS. 4B and 4C and between FIGS. 5B and 5C). At least a portion of filter 60 is typically bunched up within collection vial 1650, such as into a flower-like arrangement, from the filter's initial flat shape while disposed on the filter support.

Typically, sampling device 1620 is configured such that further proximal withdrawal of filter-withdrawal shaft 1672 out of internal plunger space 1686, while plunger head 1642 is within tubular container 1430, pulls collection vial 1650 out of internal plunger space 1686 via plunger-space proximal opening 1690 (as shown in the transitions between FIGS. 4C and 4D and between FIGS. 5C and 5D). It is noted that filter-withdrawal shaft 1672 of sampling device 1620 is not an element of filtration assembly 1624, but instead is removable therefrom, as shown in FIGS. 4D and 5D.

### EXPERIMENTAL RESULTS

The inventor performed a number of experiments using some of the techniques described herein.

Although many of the experiments described herein include testing for the presence of group A streptococcus bacteria, the inventor believes that the sample collection testing techniques used in these experiments would provide similar results for collecting specimen samples for deriving information from other bacteria and from viruses present in the throat.

### EXPERIMENT 1

### An Oral Cavity Pre-Rinse-Mediated Reduction in Non-Specific Antibody Reactivity in Clinical Gargle Samples

### Section 1: Materials and Methods

### Section 1.1: Sample Collection

Seven healthy subjects were asked to gargle 10 mL of sterile 0.9% Saline (B. Braun Melsungen AG. Melsungen, Germany, Catalog Number: 364-2828) for approximately 10 seconds, and spit the gargles out into a sterile collection cup. The subjects where then asked to perform an oral cavity rinse, wherein, subjects filled their mouth with tap water while keeping their heads tilted forward, and swished the water from cheek to cheek and in-between teeth for approximately 5-10 seconds prior to spitting out the water. Subjects then immediately performed a second oral cavity rinse, as detailed above. Subjects were then asked to perform a second gargle, as detailed above.

Section 1.2: Sample Processing

Gargle samples were transferred to concentration devices containing 1.2 μm (micron) Polyethersulfone (PES) membrane filters (Sterlitech, Kent, WA. USA, Catalog number: PES122005) cut to a diameter of 25 mm, using serological pipettes, and passed through the concentration devices. Four drops of 2M Sodium Nitrite (Consult Diagnostics, Richmond, VA, USA, Catalog Number: L060209-02) and 4 drops of 0.2M Acetic Acid (Consult Diagnostics, Richmond, VA, USA, Catalog Number: L060210-02) were added to squeezable extraction test tubes provided with the McKesson Strep A Dipstick kit (Consult Diagnostics, Richmond, VA, USA, Catalog Number: 5003), and extraction test tubes were agitated to ensure thorough mixing of reagents. Filters were then removed from the concentration devices and placed into the extraction tubes, oriented such that the filters wrapped around the inside of the extraction test tubes, with the upstream side of the filters inward facing.

Section 1.2.1: Sample Elution

The blunt ends of 10 μL (microliter) microbial loops (Heathrow Scientific, Vernon Hills, IL, USA, Catalog number: HS81121C) were inserted into the center space created by the filter's encirclement of the extraction tubes. Extraction tubes were squeezed 5-10 times, followed by the rotation of the microbial loops around the extraction tubes for 3 minutes while applying constant pressure against the walls of the extraction tubes and the filters. Extraction tubes were squeezed intermittently throughout this 3-minute period. After the first 1 minute and 30 seconds of filter manipulation as described above, the ends of the microbial loops were positioned against the filter at approximately half the total filter height, and pressed down against the side of the extraction tube to facilitate the partial folding of the filter. The remaining 1 minute and 30 seconds of filter manipulation was performed in this filter orientation.

At the completion of the 3-minute manipulation, filters were packed down to the bottom of the extraction tubes to facilitate immunoassaying using the blunt end of the microbial loops.

Section 1.2.2: Immunological Testing

McKesson Strep A Dipsticks (Consult Diagnostics, Richmond, VA, USA, Catalog Number: 5003) and OSOM Strep A Test strips (Sekisui Diagnostics, LLC., San Diego, CA, USA, Catalog number:141), immunochromatographic dipsticks specific to group A Streptococcal antigen, were inserted into extraction tubes, and removed after minutes. Strips were examined and band intensity was scored on a scale of 0-5 to determine semi-quantitative differences in antibody cross-reactivity, and any score below 1 was considered to be 0. Strips were then re-examined and re-scored after a second 5-minute wait period. In testing 3 out of the 7 paired gargle samples, following the removal of test strips, 60 μ(microliters) of the solution remaining in each of the extraction tubes was transferred directly onto the immunoassay strip of a BinaxNOW™ Strep A Card (Abbot, Scarborough, ME, USA, Catalog number: 730-025) by opening the card and pipetting into the exposed hole on the left-hand side of the card. Strips were similarly examined and re-examined 5- and 10-minutes following pipetting and band intensity was scored on a scale of 0-5 to determine semi-quantitative differences in antibody cross-reactivity, and any score below 1 was considered to be 0.

Section 2: Results and Discussion

With regards to McKesson dipsticks, in 6 out of the 7 paired samples, both gargles displayed no observable cross-reactivity. In 1 out of the 7 paired samples, the gargle sample collected prior to the oral cavity rinse exhibited higher cross-reactivity than the gargle sample collected following the oral cavity rinse

With regards to OSOM strips, in 5 out of the 7 paired samples (71% of subjects), the gargle sample collected prior to the oral cavity rinse exhibited higher cross-reactivity than the gargle sample collected following the oral cavity rinse. In 2 out of the 7 paired samples (29% of subjects), the gargle sample collected following the oral cavity rinse exhibited higher cross-reactivity than the gargle sample collected prior to the oral cavity rinse.

With regards to the BinaxNOW™ Strep A Cards, in all 3 subjects (100%), the gargle sample collected prior to the oral cavity rinse exhibited higher cross-reactivity than the gargle sample collected following the oral cavity rinse.

Obvious trends of immunoassay cross-reactivity reduction were observed following oral cavity rinsing across both OSOM and BinaxNOW™ Group A streptococcus immunoassay platforms. It is of note that even while testing with McKesson dipsticks, which appeared to have a higher specificity than the other immunoassays tested, we observed an instance were oral cavity rinsing reduced immunoassay cross-reactivity. This data supports the utilization of an oral cavity rinse prior to gargle sample collection for the reduction of non-specific antibody binding during Group A streptococcus testing. It follows that an oral cavity rinse will likewise reduce antibody reactive particles deleteriously producing false positive test results from other crude oral cavity sample types such as spit.

### EXPERIMENT 2

### Effects of Toothbrushing on Non-Specific Antibody Cross-Reactivity in Clinical Gargle Samples

Section 1: Materials and Methods

Section 1.1: Sample Collection

Two healthy subjects were asked to gargle 10 mL of sterile 0.9% Saline (B. Braun Melsungen AG. Melsungen, Germany, Catalog Number: 364-2828) for approximately 10 second, and spit the gargles out into a sterile collection cup. Subjects were then asked to brush their teeth thoroughly with tap water for approximately 2 minutes using a soft toothbrush (Orbital, Charcoal, Ultra Slim ≤0.01), ensuring they brush all accessible surfaces of their teeth. Subjects used a total of between 180-360 mL of tap water over the duration of brushing and while rinsing their teeth at the end of the brushing process. Subjects were then asked to perform a second gargle, as detailed above. One subject was resampled in the above manner the following day.

Section 1.2: Sample Processing

Gargle samples were transferred to concentration devices containing 1.2 μM (micron) Polyethersulfone (PES) membrane filters (Sterlitech, Kent, WA. USA, Catalog number: PES122005) cut to a diameter of 25 mm, using serological pipettes, and passed through the concentration devices. Four drops of 2M Sodium Nitrite (Consult Diagnostics, Richmond, VA, USA, Catalog Number: L060209-02) and 4 drops of 0.2M Acetic Acid (Consult Diagnostics, Richmond, VA, USA, Catalog Number: L060210-02) were added to squeezable extraction test tubes provided with the McKesson Strep A Dipstick kit (Consult Diagnostics, Richmond, VA, USA, Catalog Number: 5003), and extraction test tubes were agitated to ensure thorough mixing of reagents. Filters were then removed from the concentration devices and placed into the extraction tubes, oriented such that the filters wrapped around the inside of the extraction test tubes, with the upstream side of the filters inward facing.

Section 1.2.1: Sample Elution

The blunt end of 10 μL (microliter) microbial loops (Heathrow Scientific, Vernon Hills, IL, USA, Catalog number: HS81121C) were inserted into the center space created by the filter's encirclement of the extraction tubes. Extraction tubes were squeezed 5-10 times, followed by the rotation of the microbial loops around the extraction tubes for 3 minutes while applying constant pressure against the walls of the extraction tubes and the filters. Extraction tubes were squeezed intermittently throughout this 3-minute period. After the first 1 minute and 30 seconds of filter manipulation as described above, the ends of the microbial loops were positioned against the filter at approximately half the total filter height, and pressed down against the side of the extraction tube to facilitate the partial folding of the filter. The remaining 1 minute and 30 seconds of filter manipulation was performed in this filter orientation. At the completion of the 3-minute manipulation, filters were packed down to the bottom of the extraction tubes to facilitate immunoassaying using the blunt end of the microbial loops.

Section 1.2.2: Immunological Testing

OSOM Strep A Test strips (Sekisui Diagnostics, LLC., San Diego, CA, USA, Catalog number:141), immunochromatographic dipsticks specific to group A Streptococcal antigen, were inserted into extraction tubes, and removed after 5 minutes. Strips were examined and band intensity was scored on a scale of 0-5 to determine semi-quantitative differences in antibody cross-reactivity, and any score below 1 was considered to be 0. Strips were then re-examined and re-scored after a second 5 minutes wait period.

Section 2: Results and Discussion

In 2 out of 3 paired samples, gargle samples collected prior to toothbrushing exhibited higher cross-reactivity than the gargle sample collected following toothbrushing. In a single paired sample, no cross-reactivity was observed in either sample.

This data supports the use of toothbrushing prior to gargle sample collection as an effective means to reduce non-specific antibody binding during Group A streptococcus testing. It follows that toothbrushing will likewise reduce antibody reactive particles deleteriously producing false positive test results from other crude oral cavity sample types such as spit.

### EXPERIMENT 3

### The Inclusion of an Oral Cavity Pre-Rinse Does Not Adversely Affect Group A Streptococcus Detection in Clinical Gargle Samples

Section 1: Materials and Methods

Section 1.1: Sample Collection

Two Subjects diagnosed with active Group A streptococcus infections by throat culture within 24 hours prior to sampling were subjected to a second throat swab for confirmatory rapid immunological testing. Subjects thoroughly rinsed their mouths with tap water for approximately one minute, using between 720-900 mL of tap water in total. Subjects were then asked to gargle 10 mL of sterile 0.9% Saline (B. Braun Melsungen AG. Melsungen, Germany, Catalog Number: 364-2828) for approximately second, and spit the gargles out into a sterile collection cup.

Section 1.2: Sample Processing

Gargle samples were transferred to concentration devices containing 1.2 μM (micron) Polyethersulfone (PES) membrane filters (Sterlitech, Kent, WA. USA, Catalog number: PES122005) cut to a diameter of 25 mm, using serological pipettes, and passed through the concentration devices. Four drops of 2M Sodium Nitrite (Consult Diagnostics, Richmond, VA, USA, Catalog Number: L060209-02) and 4 drops of 0.2M Acetic Acid (Consult Diagnostics, Richmond, VA, USA, Catalog Number: L060210-02) were added to squeezable extraction test tubes provided with the McKesson Strep A Dipstick kit (Consult Diagnostics, Richmond, VA, USA, Catalog Number: 5003), and extraction test tubes were agitated to ensure thorough mixing of reagents. Filters were then removed from the concentration devices and placed into the extraction tubes, oriented such that the filters wrapped around the inside of the extraction test tubes, with the upstream side of the filters inward facing.

Section 1.2.1: Sample Elution

The blunt ends of 10 μL (microliter) microbial loops (Heathrow Scientific, Vernon Hills, IL, USA, Catalog number: HS81121C) were inserted into the center space created by the filter's encirclement of the extraction tubes. Extraction tubes were squeezed 5-10 times, followed by the rotation of the microbial loops around the extraction tubes for 3 minutes while applying constant pressure against the walls of the extraction tubes and the filters. Extraction tubes were squeezed intermittently throughout this 3-minute period. After the first 1 minute and 30 seconds of filter manipulation as described above, the ends of the microbial loops were positioned against the filter at approximately half the total filter height, and pressed down against the side of the extraction tube to facilitate the partial folding of the filter. The remaining 1 minute and 30 seconds of filter manipulation was performed in this filter orientation. At the completion of the 3-minute manipulation, filters were packed down to the bottom of the extraction tubes to facilitate immunoassaying using the blunt end of the microbial loops.

Section 1.2.2: Immunological Testing

McKesson Strep A Dipsticks (Consult Diagnostics, Richmond, VA, USA, Catalog Number: 5003) and OSOM Strep A Test strips (Sekisui Diagnostics, LLC., San Diego, CA, USA, Catalog number:141), immunochromatographic dipsticks specific to group A Streptococcal antigen, were inserted into extraction tubes, and removed and examined after 5 minutes.

Section 2: Results and Discussion

Immunoassay results from gargle samples produced from both subjects were concordant with throat culture results across both immunoassay testing platforms (McKesson Strep A Dipstick, and OSOM Strep A Test), suggesting that the incorporation of an oral cavity rinse prior to gargle sampling does not adversely affect diagnostic sensitivity. Taken together with results from Experiment 1 (above), the inventor hypothesizes that the efficacy of Group A Streptococcus diagnostic devices testing whole mouth samples such as gargle or spit could be greatly increased with the inclusion of an oral cavity rinse, by increasing specificity without negatively impacting sensitivity. Furthermore, the utilization of an oral cavity rinse prior to sampling could allow for the commercialization of more sensitive Immunoassay tests due to the reversal of specificity-limited design constraints.

### EXPERIMENT 4

### Methodologies for Reducing the Amount of Particulates Cross-Reactive with Anti-Streptococcal Antibodies Associated with the Teeth of Healthy Individuals

Section 1: Materials and Methods

Section 1.1: Sample Collection

Thirteen healthy subjects were subjected to teeth swabbing, in which sterile rayon swabs (Puritan Medical Products Company LLC, Guilford, ME, USA, Catalog number: 25-806 1PR) were rubbed against all accessible surfaces of the subjects' teeth, as well as the gingiva at regions close to the junction between the gingiva and the teeth.

Twelve of the 13 subjects were then asked to brush their teeth using one of three toothpastes; Colgate SMILES (Colgate-Palmolive, Sidnica, Poland), Colgate Optic WHITE (Colgate-Palmolive, Sidnica, Poland), or Taftapim Children Toothpaste with Grape Flavor (Hod Hasharon, Israel), wherein 1 subject used Taftapim Children Toothpaste with Grape Flavor, 3 subjects used Colgate SMILES, and 8 subjects used Colgate Optic WHITE. The duration of toothbrushing was approximately 2 minutes, during which subjects were asked to thoroughly brush all accessible surfaces of the teeth, followed by a tap-water cleaning period wherein subjects were asked to ensure that no visible toothpaste remained in their mouth. Two of the 12 subjects then immediately performed a second subsequent toothbrushing. The single subject that did not perform a toothbrushing instead performed an Oral Cavity Rinse, as detailed in Experiment 1, Section 1.1. All subjects were then subjected to a second teeth swabbing, as detailed above.

Section 1.2: Sample Processing and Elution

Four drops of 2M Sodium Nitrite (Consult Diagnostics, Richmond, VA, USA, Catalog Number: L060209-02) and 4 drops of 0.2M Acetic Acid (Consult Diagnostics, Richmond, VA, USA., Catalog Number: L060210-02) were added to squeezable extraction test tubes provided with the McKesson Strep A Dipstick kit (Consult Diagnostics, Richmond, VA, USA., Catalog Number: 5003), and extraction test tubes were agitated to ensure thorough mixing of reagents. Teeth swabs were inserted into the extraction test tubes, in a swab-head down orientation. Teeth swabs were rotated while the extraction test tubes were squeezed approximately 5-10 times, and the swabs were removed and reinserted into the extraction test tubes approximately 2 times while squeezing, followed by an approximate 3-minute incubation period with the swab heads remaining in the extraction test tubes, with occasional swab rotation while squeezing.

Section 1.3: Immunological Testing

Teeth swabs were removed from the extraction test tubes while pinching the tubes to extract absorbed liquid, and OSOM Strep A Test strips (Sekisui Diagnostics, LLC., San Diego, CA, USA, Catalog number:141), immunochromatographic dipsticks specific to group A Streptococcal antigen, were inserted into extraction tubes, and removed after 5 minutes. Strips were examined and band intensity was scored on a scale of 0-5 to determine semi-quantitative differences in antibody cross-reactivity, and any score below 1 was considered to be 0. Strips were then re-examined and re-scored after a second 5-minute wait period.

Section 2: Results and Discussion

All subjects tested had a noticeable reduction in antibody cross-reactivity to teeth swabs following treatment (i.e., either toothbrushing or oral cavity rinsing) compared to before treatment. This demonstrates that (i) anti-group A streptococcus antibody cross-reactive material is found on the teeth and gingiva of healthy individuals, and (ii) both toothbrushing and oral cavity rinsing are effective means of reducing the amount of anti- group A streptococcus antibody cross-reactive material found on teeth and gingiva. It follows that the teeth and gingiva are likely sources of anti- group A streptococcus antibody cross-reactive material that are present in gargle samples, as described in Experiments 1 and 2.

In some applications, any of the techniques described in the report of these Experiments are used in combination with the other techniques described herein.

In an embodiment, the techniques and apparatus described herein are combined with techniques and apparatus described in one or more of the following patent applications, which are assigned to the assignee of the present application and are incorporated herein by reference: PCT Publication WO 2018/158768 to Fruchter et al.; US Patent Application Publication 2019/0381498 to Fruchter et al.; U.S. Provisional Application 62/727,208, filed Sep. 5, 2018; U.S. Provisional Application 62/727,268, filed Sep. 5, 2018; PCT Publication WO 2020/049566 to Fruchter et al.; PCT Publication WO 2020/049569 to Fruchter et al., and U.S. application Ser. No. 17/270,544 in the national stage thereof; US Patent Application Publication 2021/0102876 to Fruchter et al. U.S. Provisional Application 62/896,295, filed Sep. 5, 2019 U.S. Provisional Application 62/988,145, filed Mar. 11, 2020; U.S. Provisional Application 62/988,259, filed Mar. 11, 2020; U.S. Provisional Application 63/020,723, filed May 6, 2020; U.S. Provisional Application 63/037,707, filed Jun. 11, 2020; U.S. Provisional Application 63/067,535, filed Aug. 19, 2020; U.S. Provisional Application 63/071,529, filed Aug. 28, 2020; U.S. Provisional Application 63/117,294, filed Nov. 23, 2020; U.S. Provisional Application 63/134,282, filed Jan. 6, 2021; U.S. Provisional Application 63/156,843, filed Mar. 4, 2021; U.S. Provisional Application 63/158,005, filed Mar. 8, 2021; U.S. Provisional Application 63/166,378, filed Mar. 26, 2021; U.S. Provisional Application 63/176,565, filed Apr. 19, 2021; PCT Publication WO 2021/044417 to Holtz et al; PCT Publication WO 2021/181338 to Fruchter et al., and U.S. application Ser. No. 17/801,452 in the national stage thereof; PCT Publication WO 2021/181339 to Feldman et al.; PCT Publication WO 2021/224925 to Levitz et al., and U.S. application Ser. No. 17/921,672 in the national stage thereof; U.S. Provisional Application 63/275,130, filed Nov. 3, 2021; PCT Publication WO 2022/044002 to Levitz et al.; International Patent Application PCT/IL2022/050024, filed Jan. 6, 2022, which published as PCT Publication WO 2022/149135 to Feldman et al.; U.S. Provisional Application 63/350,521, filed Jun. 9, 2022; U.S. application Ser. No. 17/980,200, filed Nov. 3, 2022; U.S. Provisional Application 63/277,238, filed Nov. 9, 2021; U.S. Provisional Application 63/283,577, filed Nov. 29, 2021; International Patent Application PCT/IL2022/051264, filed Nov. 11, 2022.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
rinsing an oral cavity of a subject with a rinse liquid;
thereafter, introducing into the oral cavity a gargle liquid and gargling the gargle liquid by the subject by holding the gargle liquid in a throat of the subject and agitating with air expelled from lungs of the subject; and
thereafter, collecting at least a portion of the gargled liquid as a specimen sample, wherein rinsing the oral cavity removes saliva from the oral cavity prior to
gargling, thereby reducing the amount of saliva in the gargled liquid that is collected as the specimen sample.

2. The method according to claim 1, wherein the rinse liquid has a pH of 5-9.5.

3. The method according to claim 2, wherein the rinse liquid has a pH of 5-7.

4. The method according to claim 2, wherein the rinse liquid has a pH of 6-8.5.

5. The method according to claim 1, wherein rinsing the oral cavity with the rinse liquid comprises drawing the rinse liquid from a faucet connected to a public water supply.

6. The method according to claim 1, wherein the rinse liquid is tap water.

7. The method according to claim 1, wherein the gargle liquid has a salt concentration of 0-0.154 mol/L.

8. The method according to claim 1, wherein the gargle liquid has a sweet taste.

9. The method according to claim 1, wherein the rinse liquid does not comprise a drug molecule.

10. The method according to claim 1, wherein the gargle liquid does not comprise a drug molecule.

11. The method according to claim 1, wherein the gargle liquid does not include a tastant.

12. The method according to claim 1, wherein the gargle liquid has a salty taste or does not include a tastant.

13. The method according to claim 1, wherein the method does not comprise brushing teeth of the subject.

14. The method according to claim 1, further comprising, before rinsing the oral cavity, cleaning teeth of the subject.

15. The method according to claim 14, wherein cleaning the teeth comprises brushing the teeth.

16. The method according to claim 15, wherein brushing the teeth comprises brushing the teeth using a toothbrush having soft bristles.

17. The method according to claim 15, wherein brushing the teeth comprises brushing the teeth with toothpaste.

18. The method according to claim 15, wherein brushing the teeth comprises brushing the teeth without toothpaste.

19. The method according to claim 1, further comprising after collecting the specimen sample, testing the specimen sample for the presence of one or more particulates.

20. The method according to claim 19, wherein testing the specimen sample for the presence of the one or more particulates comprises diagnosing a disease.

21. The method according to claim 19, wherein testing the specimen sample for the presence of the one or more particulates comprises concentrating the specimen sample by filtration, by passing the specimen sample through one or more filters.

22. The method according to claim 19, wherein testing the specimen sample for the presence of the one or more particulates comprises performing an immunoassay using a lateral flow immunoassay test strip.

23. The method according to claim 19, wherein the one or more particulates are selected from the group consisting of: a pathogenic microorganism, a non-pathogenic microorganism, an antigen, a human cell, a cellular biomarker, a hormone, a chemical mediator from a cell, a pollen, a nucleic acid, and a chemical originating from an external vapor.

24. The method according to claim 19, wherein the one or more particulates are selected from the group consisting of: a virus, a bacterium, a protozoan, and a fungus.

25. The method according to claim 24, wherein the one or more particulates include the bacterium.

26. The method according to claim 25, wherein the bacterium is a Streptococcus bacterium.

27. The method according to claim 24, wherein the one or more particulates include a virus.

28. The method according to claim 27, wherein the virus is selected from the group of viruses consisting of: an Influenza virus, Epstein-Barr virus, and respiratory syncytial virus (RSV).

29. The method according to claim 1, wherein introducing the gargle liquid into the oral cavity comprises introducing the gargle liquid into the oral cavity within 5 minutes of finishing rinsing the oral cavity, thereby limiting the amount of saliva that reaccumulates in the oral cavity after rinsing.

30. (canceled)

31. The method according to claim 1, wherein rinsing the oral cavity comprises rinsing teeth and gingiva of the subject, including in regions between the teeth and a cheek of the subject and between the teeth and inner surfaces of lips of the subject.
